(19) United States
(12) Patent Application Publication
(10) Pub. No.: US 2004/0038937 A1
(43) Pub. Date: Feb. 26, 2004

(54) Method and device for avoiding alteration of a substance having biological activities

(76) Inventors: Jacques Benveniste, Paris (FR); Didier Guillonnet, Cagnes-sur-Mer (FR)

Correspondence Address: Robert M Angus, Westman Champlin & Kelly, International Centre Suite 1600, 900 Second Avenue South, Minneapolis, MN, 55402-3319, US

(21) Appl. No.: 10/332,609

(22) PCT Filed: Jul. 5, 2001
(86) PCT No.: PCT/FR01/02172

(30) Foreign Application Priority Data
Jul. 12, 2000 (FR) 00/09169

(51) Int. Cl. H05K 9/00; A61K 31/727; A61K 9/22

(52) U.S. Cl. 514/56; 604/890.1

(57) ABSTRACT

The invention concerns a method applied to a substance treated to exhibit a biological activity, for example a coagulating or anticoagulation activity. The treated substance has been obtained from a source substance having the biological activity, after a treatment such that the treated substance does not contain any molecule of the source substance in significant amount. The treatment can in particular consist in using a high dilution process of the same type used for producing homeopathic solutions or granules. The method is designed to protect the treated substance from external factors to which it is subjected and is characterised in that it comprises the step which consists in placing the treated substance in a chamber (19), sheltered from electromagnetic fields. The chamber (19) is enclosed with a magnetic shield (19a) made in particular of soft iron or mumetal.

It has been described in patent N° WO 00/17638, published on Mar. 30, 2000 and entitled “Method, system and device for producing from a substance signals, in particular electrical signals, characteristic of the biological and/or chemical activity of said substance,” that a receiving substance initially not having any particular biological activity, in particular water, can be treated in such a fashion that it exhibits, after treatment, biological activity. After treatment, the receiving substance is called the “Treated Substance” (or alternatively the Informed Matter). When the receiving substance is water, the Treated Substance is called “Treated Water” (or alternatively “Informed Water”). The substance having a biological activity can also take the form of a homeopathic preparation or granules.

It will be recalled, later on in the description of the present invention, how the Informed Water can be produced using a mother substance, in particular a mother substance having an anticoagulant effect, such as heparin. This recollection will be made with reference to patent N° WO 00/17637 published on Mar. 30, 2000 and entitled “Method and system for producing a substance or a signal having a coagulant or an anticoagulant effect and therapeutic applications of said substance or said signal.”

Although this has been described in this same patent N° WO 00/17637 for testing a Treated Substance, in particular Informed Water, it is applied to a sensitive biological system in order to observe the effects. For example, in the case wherein Informed Water having a coagulant or an anticoagulant effect is tested, the Informed Water and the plasma are mixed in the following proportions (⅓, ⅔), respectively. Then the coagulation times are measured.

Such tests are particularly sensitive to perturbation phenomena. The inventors have been able to confirm, surprisingly and inexplicably, that certain individuals have an inhibitor or potentiator effect on the Informed Water. For example, they need merely approach informed water to alter the properties thereof. However, it is not possible to know a priori which people have such inhibitor effects. The transport and manipulation by such people of Treated Substances is an obstacle (the activity is altered) to the industrial development of these substances. Thus we are faced with the problem: how to prevent perturbations from modifying the activity of a Treated Substance.

There is no known prior art that proposes a solution to this problem. The reason for this is simple: on the one hand, until the research done by the inventors, it was not known that the activity of water could be changed and, on the other hand, there was no awareness that unknown phenomena could alter, at a distance, the biological properties of Informed Water or homeopathic granules.

The invention relates to a method for preventing the alteration of a substance having the biological activities of a source substance no longer present in a significant quantity.

The method is applied to a substance having a biological activity, for example, a coagulant or an anticoagulant activity. The substance has been obtained using a source substance having the biological activity, at the end of a treatment such that the substance does not contain any molecules of the source substance in a significant quantity (the substance is hereinafter called the treated substance). The treatment can consist in particular in implementing a high dilution method of the same type as that used to produce homeopathic solutions or granules. The treatment can also consist in particular in implementing the method according to Jacques Benveniste, comprising several steps as described in patent N° WO 00/17637 published on Mar. 30, 2000 and entitled “Method and system for producing a substance or a signal having a coagulant or an anticoagulant effect and therapeutic applications of said substance or said signal”. It comprises the step of transforming the electromagnetic field originating from the source substance having a biological activity into a signal, in particular an electrical signal, by means of a transducer-receiver picking up the electromagnetic field. The source substance has, for example, an effect on the coagulation of blood and can, for example, contain Ca⁺⁺ ions or heparin. The method comprises, in addition, the step of applying to a receiving substance, not initially having any particular biological activity, the signal originating from the transducer-receiver by means of a transducer-emitter. Thus, for example, after the treatment defined hereinabove, the receiving substance, not initially having any particular biological activity, then has a coagulant or an anticoagulant activity.

The method has been conceived to protect the treated substance from the external influences, to which it is subjected, and is characterized in that it comprises the step of placing the treated substance into a receptacle protected from electromagnetic fields.

Preferably, the receptacle is surrounded by a magnetic shielding made of soft iron or mumetal.

The invention also relates to a device applied to a substance having a biological activity, for example a coagulant or an anticoagulant activity. The substance has been obtained using a source substance having the biological activity, at the end of a treatment such that the substance does not contain any molecules of the source substance in a significant quantity (the substance is hereinafter called the treated substance). The treatment can consist in particular in implementing a high dilution method of the same type as that used to produce homeopathic solutions or granules. The treatment can also consist in particular in implementing the method according to Jacques Benveniste, comprising several steps as described in patent N° WO 00/17637 published on Mar. 30, 2000 and entitled “Method and system for producing a substance or a signal having a coagulant or an anticoagulant effect and therapeutic applications of said substance or said signal.” It comprises the step of transforming the electromagnetic field originating from the source substance having a biological activity into a signal, in particular an electrical signal, by means of a transducer-receiver picking up the electromagnetic field. The source substance has, for example, an effect on the coagulation of blood and can, for example, contain Ca⁺⁺ ions or heparin. It comprises, in addition, the step of applying to a receiving substance, not initially having any particular biological activity, the signal originating from the transducer-receiver by means of a transducer-emitter.

Thus, for example, after the treatment defined hereinabove, the receiving substance, not initially having any particular biological activity, now has a coagulant or an anticoagulant activity.

The method has been conceived to protect the treated substance from the external influences, to which it is subjected, and is characterized in that it comprises the step of placing the treated substance in a receptacle, protected from electromagnetic fields.

Preferably, the receptacle is surrounded by a magnetic shielding made of soft iron or mumetal.

Other characteristics and advantages of the invention will become apparent on reading the description of the various embodiments given as indicative and non-limiting examples

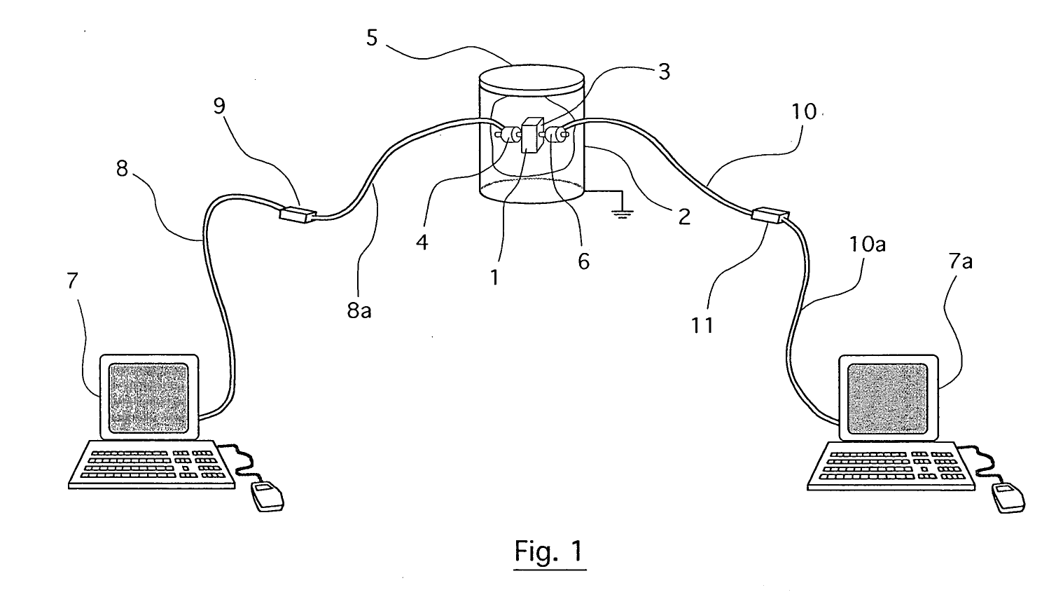

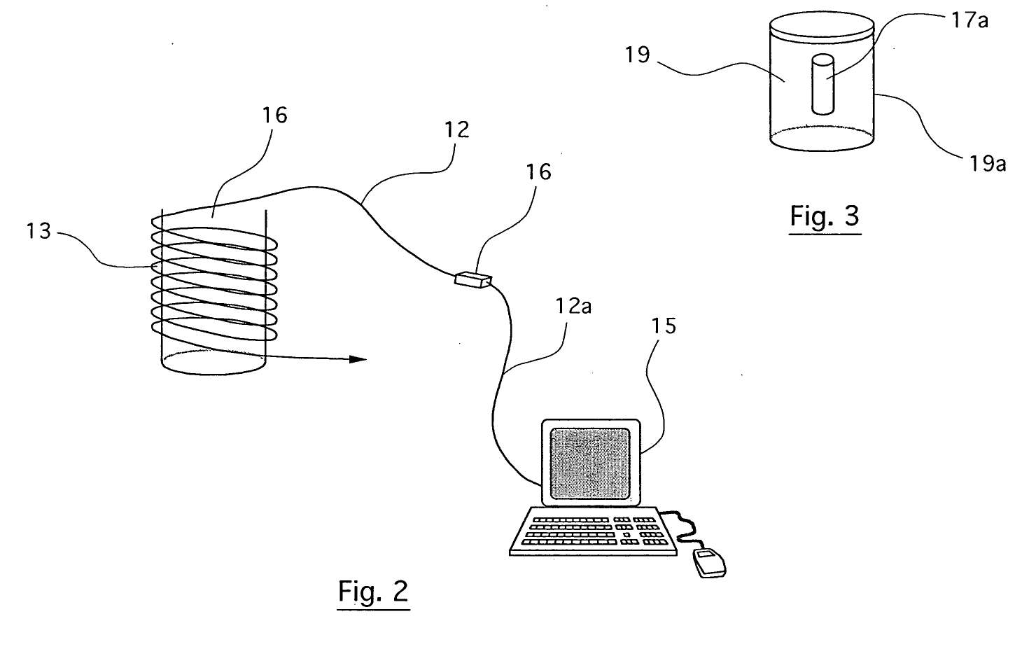

and referring to FIGS. 1 and 2 depicting a system enabling the production of “informed water” using a mother substance having an anticoagulant effect, in particular heparin,

and referring to FIG. 3 depicting a schematic illustration of the method and the device according to the invention enabling the alteration of a substance having biological activities to be avoided.

First of all, it will be recalled how the “informed water” can be produced using a mother substance having an anticoagulant effect, in particular heparin. FIGS. 1 and 2 depict a system conforming to that described in patent N° WO 00/17637 published on Mar. 30, 2000 and entitled “Process and system for producing a substance or a signal having a coagulant or an anticoagulant effect and therapeutic applications of said substance or said signal.” The system makes it possible to produce, using a mother substance, electrical signals characteristic of its biological activity. In the present instance, the mother substance is heparin 1 having an anticoagulant effect. The system comprises a cylindrical receptacle 2 equipped with a shielding insulating the receptacle 2 from parasitic fields originating from the environment. The receptacle 2 is closed by a cover 5 that is likewise shielded. An emitter 4 is disposed on the inside of the receptacle. It generates a predetermined electromagnetic excitation field. The emitter is supplied by a generator 7 conceived for generating a low frequency signal of pink noise or, advantageously, white noise. As has been indicated, the generator 7 takes the form of a microcomputer 7 equipped with a sound card comprising an analog-digital converter. The converter comprises an output terminal 8. The output terminal 8 of the sound card of the microcomputer 7 is connected to the input terminal 8a of the emitter 4 via an amplifier 9. A transducer-receiver 6 as well as a plastic cup 3 containing 1 ml of heparin is placed in the receptacle 2. The transducer-receiver 6 receives the fields resulting from the interaction of the specific excitation field and the heparin 1. The transducer-receptor 6 transforms these fields into electrical signals. These electrical signals are present at the output 10 of the transducer-receiver 6 in the form of a variable potential difference or an electrical current of variable intensity. The electrical signals available at the output 10 of the transducer-receiver 6 are amplified by a pre-amplifier 11. The electrical signals, after analog-digital conversion, are subject to digital recording. For this purpose, a microcomputer 7a of the same type as the microcomputer 7 is used, comprising an analog-digital converter. The output 10 of the transducer-receiver 6 is connected to the input 10a of the analog-digital converter of the microcomputer 7a by way of the pre-amplifier 11. In addition, as has been represented in FIG. 2, the system makes it possible to produce “informed water” using electrical signals obtained as described hereinabove. A receiving substance not initially having any particular biological activity, in particular water, is placed in a plastic cup 14. Said cup 14 is disposed in an electromagnetic field radiated by a transducer-emitter 13, in the present case a coil 13. The input terminals 12 of the coil 13 are connected to the output 12a of an analog-digital converter of a microcomputer 15 by way of an amplifier 16. The microcomputer 15 contains in the memory the electrical signals digitized by means of the microcomputer 7. The microcomputer 15 comprising the analog-digital converter is similar to the microcomputer 7 described hereinabove. The analog-digital converter produces electrical signals using the digital recording. Thus, by applying the electrical signals to the receiving substance contained in the cup 14 by means of the transducer-emitter 13, informed water is obtained that has an anticoagulant activity. The informed water obtained in this fashion is capable of industrial applications insofar as its properties are not altered. As has been stated in the introduction of this present description, certain individuals have an inhibitor or, conversely, a potentiator effect on informed water, in particular when handling a tube containing informed water.

FIG. 3 depicts a schematized illustration of the method and of the system according to the invention which make it possible to prevent the alteration of a substance having biological activities. “Potential alterations” are designated as being the inhibitor or potentiator effect that an individual can produce on informed water by handling the tube containing it or by approaching close to the tube. A tube 17a contains informed water having an anticoagulant activity. The informed water has been previously produced as described hereinabove. In order to prevent alteration of the informed water, the tube 17a containing it is placed in a receptacle 19 in order to protect it from electromagnetic fields. The receptacle 19 is surrounded by a magnetic shielding 19a made of soft iron or mumetal. Thus, the tube 17a is arranged protected from potential alterations, in particular the alterations by an individual producing an inhibitor or potentiator effect on informed water.

1. A method applied to a substance having a biological activity, for example a coagulant or an anticoagulant activity;
said substance having been obtained using a source substance (1) having said biological activity, at the end of a treatment such that the substance does not contain any molecules of the source substance (1) in a significant quantity (said substance being hereinafter called the treated substance);
said treatment consisting in particular in implementing a high dilution method of the same type as that used to produce homeopathic solutions or granules,
said treatment also possibly consisting in particular in implementing the method according to Jacques Benveniste comprising the following steps:
the step of transforming the electromagnetic field originating from said source substance (1) having a biological activity;
(for example, originating from a source substance having an effect on the coagulation of blood such as a solution containing in particular Ca⁺⁺ ions or heparin),
into a signal, in particular an electrical signal, by means of a transducer-receptor (6) picking up said electromagnetic field;
the step of applying to a receiving substance said signal originating from said transducer-receiver (6) by means of a transducer-emitter (13),
(in such a fashion that, for example, after the treatment defined hereinabove, the receiving substance, initially inactive, has a coagulant or an anticoagulant activity);
said method being conceived to protect the treated substance from the external influences, to which it is subjected, and characterized in that it comprises the step of placing the treated substance into a receptacle (19) protected from electromagnetic fields.

2. A method according to claim 1, wherein the receptacle (19) is surrounded by a magnetic shielding (19a) made of, in particular, soft iron or mumetal.

3. A device applied to a substance having a biological activity, for example a coagulant or an anticoagulant activity;
said substance having been obtained using a source substance (1) having said biological activity, at the end of a treatment such that the substance does not contain any molecules of the source substance (1) in a significant quantity (said substance being called hereinafter the treated substance);
said treatment possibly consisting in particular in implementing a high dilution method of the same type as that used to produce homeopathic solutions or granules,
said treatment also possibly consisting in particular in implementing the method according to Jacques Benveniste comprising the following steps:
the step of transforming the electromagnetic field originating from said source substance (1) having a biological activity,
(for example, originating from a source substance having an effect on the coagulation of blood such as a solution containing in particular Ca⁺⁺ ions or heparin),
into a signal, in particular an electrical signal, by means of a transducer-receiver (6) picking up said electromagnetic field;
the step of applying to a receiving substance said signal originating from said transducer-receiver (6) by means of a transducer-emitter (13),
(in such a fashion that, for example, after the treatment defined hereinabove, the receiving substance, initially inactive, has a coagulant or anticoagulant activity);
said device being conceived to protect the treated substance from the external influences, to which it is subjected, in particular the influence of people who are situated in proximity to the treated substance; said device being characterized in that it comprises a receptacle (19) comprising a housing for containing the treated substance protected from electromagnetic fields.

4. A device according to claim 3, wherein the receptacle (19) is surrounded by a magnetic shielding (19a) made in particular of soft iron or mumetal.
